(19) United States
(12) Patent Application Publication
(10) Pub. No.: US 2003/0104454 A1
(43) Pub. Date: Jun. 5, 2003

(54) Method for detection of DNA methyltransferase RNA in plasma and serum

(76) Inventor: Michael S. Kopreski, Long Valley, NJ (US)

Correspondence Address: MCDONNELL BOEHNEN HULBERT & BERGHOFF, 300 SOUTH WACKER DRIVE, SUITE 3200, CHICAGO, IL, 60606, US

(21) Appl. No.: 10/288,935

(22) Filed: Nov. 5, 2002

Related U.S. Application Data
(60) Provisional application No. 60/332,622, filed on Nov. 5, 2001.

(51) Int. Cl. C12Q 1/68; C12P 19/34

(52) U.S. Cl. 435/6; 435/91.2

(57) ABSTRACT

The methods of the invention detect in a qualitative or quantitative fashion DNA methyltransferase RNA in blood plasma, serum, and other bodily fluids. The inventive methods are useful for aiding detection, diagnosis, monitoring, treatment, or evaluation of neoplastic disease, and for identifying individuals who have a predisposition to disease or who might benefit from further evaluation, monitoring or therapy.

This application claims priority to U.S. Provisional Application Serial No. 60/332,622 filed Nov. 5, 2001.

### BACKGROUND OF THE INVENTION

This invention relates to methods for detecting and monitoring DNA methyltransferase RNA (DNMT RNA) in bodily fluids such as blood plasma and serum. DNA methylation appears to play an important role in the pathogenesis and maintenance of cancer. Hypermethylation of tumor-associated genes such as tumor suppressor genes, and hypomethylation of other genomic DNA, are frequently observed in cancer. DNA methylation is to a large extent regulated by DNA methyltransferases, of which three DNA methyltransferase gene families are DNA methyltransferasel (DNMT1), DNA methyltransferase 2 (DNMT2), and DNA methyltransferase 3 (DNMT3). DNMT is thus expressed in many malignant and premalignant tissues, wherein in particular DNMT1 and DNA methyltransferase 3a (DNMT3a) and DNA methyltransferase 3b (DNMT3b), and particularly DNMT3b, is over-expressed in malignancy. Since ribonucleic acid (RNA) is essential for producing DNMT protein, detection and monitoring of DNA methyltransferase RNA (DNMT RNA) provides a method for assessing and monitoring DNMT gene expression, thereby providing a method for monitoring and determining the presence and propensity for aberrant DNA methylation. DNMT RNA is associated with cancer and premalignancy, and is therefore characterized as a tumor-associated RNA herein.

Co-owned and co-pending U.S. Patent Application Serial No. 09/155,152, incorporated herein by reference in its entirety, provides a method for detecting tumor-associated RNA in bodily fluids such as blood plasma and serum, wherein said RNA detection is used for detecting, monitoring, or evaluating cancer or premalignant conditions.

DNMT RNA being recognized herein as being tumor-associated RNA, there is a newly-appreciated need in the art to identify premalignant or malignant states in an animal, most preferably a human, by detecting in a qualitative or quantitative fashion DNMT RNA such as DNMT1 RNA, DNMT3a RNA, and DNMT3b RNA in bodily fluids such as blood plasma or serum. Further, there is a need in the art to evaluate the predisposition in an animal, most preferably a human, for diseases characterized by aberrant DNA methylation, including DNA hypermethylation and hypomethylation, by detecting DNMT RNA in bodily fluids such as blood plasma or serum, wherein cancer is one such disease.

### SUMMARY OF THE INVENTION

The present invention describes a method of evaluating an animal, most preferably a human, for premalignant or malignant states, disorders or conditions by detecting DNMT mRNA in bodily fluids, preferably blood and most preferably blood plasma and serum as well as in other bodily fluids, preferably urine, effusions, ascites, saliva, cerebrospinal fluid, cervical, vaginal, and endometrial secretions, gastrointestinal secretions, bronchial secretions, and associated tissue washings and lavages. Specific DNMT RNAs are recognized to include DNMT1 RNA, DNMT2 RNA, DNMT3a RNA, and DNMT3b RNA.

The invention provides the method of amplifying and detecting extracellular DNMT RNA. In a preferred embodiment, the present invention provides a method for detecting DNMT RNA in blood or a blood fraction, including plasma and serum, or in other bodily fluids, the method comprising the steps of extracting RNA from blood, plasma, serum, or other bodily fluid, in vitro amplifying in a qualitative or quantitative fashion one or more DNMT mRNA or their cDNA, and detecting the amplified product of DNMT mRNA or its cDNA. Said amplification methods may further include the qualitative or quantitative comparison to a reference RNA species normally present in the plasma, serum, or bodily fluid of individuals with or without cancer.

In a first aspect of this embodiment, the present invention provides methods for detecting DNMT RNA in blood or blood fractions, including plasma and serum, in a human or animal. Said methods are useful for detecting, diagnosing, monitoring, treating and evaluating various proliferative disorders, particularly stages of neoplastic disease, including premalignancy, early cancer, non-invasive cancer, carcinoma in-situ, invasive cancer and advanced cancer. In this aspect, the method comprises the steps of extracting RNA from blood or blood plasma or serum, in vitro amplifying said DNMT RNA comprising the extracted RNA either qualitatively or quantitatively, and detecting the amplified product of DNMT RNA or its cDNA.

The invention in a second aspect provides a method for detecting DNMT RNA in any bodily fluid. Preferably, said bodily fluid is whole blood, blood plasma, serum, urine, effusions, ascitic fluid, saliva, cerebrospinal fluid, cervical secretions, vaginal secretions, endometrial secretions, gastrointestinal secretions, bronchial secretions including sputum, secretions or washings from the breast, or other associated tissue washings or lavages from a human or animal. In this aspect, the method comprises the steps of extracting RNA from the bodily fluid, in vitro amplifying DNMT RNA comprising a fraction of the extracted RNA, or preferably the corresponding cDNA into which the RNA is converted, in a qualitative or quantitative fashion, and detecting the amplified product of DNMT RNA or cDNA. In these embodiments, the inventive methods are particularly advantageous for detecting, diagnosing, monitoring, treating or evaluating various proliferative disorders, particularly stages of neoplastic disease, including premalignancy, early cancer, non-invasive cancer, carcinoma-in-situ, invasive cancer and advanced cancer.

The method of the invention is additionally useful for identifying DNMT RNA- expressing cells or tissue in an animal, most preferably a human. In these embodiments, detection of an in vitro amplified product of DNMT RNA using the inventive methods indicates the existence of DNMT RNA-expressing cells or tissue in an animal, most preferably a human.

The invention provides primers and probes useful in the efficient amplification of extracellular DNMT mRNA or cDNA from bodily fluid, most preferably blood plasma or serum.

The invention further provides a diagnostic kit for detecting DNMT RNA in bodily fluid, preferably blood plasma or serum, wherein the kit comprises primers, probes or both primers and probes for amplifying and detecting extracellular DNMT RNA or cDNA derived therefrom.

In preferred embodiments of the inventive methods, DNMT RNA is extracted from whole blood, blood plasma or serum, or other bodily fluids using an extraction method such as but not limited to gelatin extraction method; silica, glass bead, or diatom extraction method; guanidinium thiocyanate acid-phenol based extraction methods; guanidinium thiocyanate acid based extraction methods; methods using centrifugation through cesium chloride or similar gradients; phenol-chloroform based extraction methods; or other commercially available RNA extraction methods. Extraction may further be performed using probes that specifically hybridize to DNMT RNA.

In preferred embodiments of the inventive methods, DNMT RNA or cDNA derived therefrom is amplified using an amplification method such as reverse transcriptase polymerase chain reaction (RT-PCR); ligase chain reaction; signal amplification such as DNA signal amplification; amplifiable RNA reporters; Q-beta replication; transcription-based amplification; isothermal nucleic acid sequence based amplification; self-sustained sequence replication assays; boomerang DNA amplification; strand displacement activation; cycling probe technology; or any combination or variation thereof.

In preferred embodiments of the inventive methods, detecting an amplification product of DNMT RNA or DNMT cDNA is accomplished using a detection method such as gel electrophoresis; capillary electrophoresis; conventional enzyme-linked immunosorbent assay (ELISA) or modifications thereof, such as amplification using biotinylated or otherwise modified primers; nucleic acid hybridization using specific, detectably-labeled probes, such as fluorescent-, radioisotope-, or chromogenically-labeled probe; Northern blot analysis; Southern blot analysis; electrochemiluminescence; reverse dot blot detection; and high-performance liquid chromatography.

In particularly preferred embodiments of the inventive methods, DNMT RNA is converted to cDNA using reverse transcriptase following extraction of RNA from a bodily fluid and prior to amplification.

The methods of the invention are advantageously used for providing a diagnosis or prognosis of, or as a predictive indicator for determining a risk for an animal, most preferably a human, for developing a proliferative, premalignant, neoplastic or malignant disease comprising or characterized by the existence of cells over expressing DNMT RNA. The methods of the invention are particularly useful for providing a diagnosis for identifying humans at risk for developing or who have developed malignancy or premalignancy and for determining predisposition to malignancy. Most preferably, the malignant or premalignant diseases, conditions or disorders advantageously detected, diagnosed, or inferred using the methods of the invention are breast, prostate, ovarian, lung, cervical, colorectal, gastric, hepatocellular, pancreatic, bladder, endometrial, kidney, skin, and esophageal cancers, and premalignancies and carcinoma in-situ such as prostatic intraepithelial neoplasia (PIN), cervical dysplasia, cervical intraepithelial neoplasia (CIN), bronchial dysplasia, atypical hyperplasia of the breast, ductal carcinoma in-situ, colorectal adenoma, atypical endometrial hyperplasia, and Barrett's esophagus.

In certain preferred embodiments of the methods of the invention, DNMT RNA or cDNA derived therefrom is amplified in a quantitative manner, thereby enabling the quantitative comparison of DNMT RNA present in a bodily fluid such as blood plasma or serum from an animal, most preferably a human. In these embodiments, the amount of extracellular DNMT RNA detected in an individual are compared with a range of amounts of extracellular DNMT RNA detected in said bodily fluid in populations of animals known to have a premalignant, neoplastic, or malignant disease, most preferably a particular premalignant, neoplastic, or malignant disease. Additionally, the amount of extracellular DNMT RNA detected in an individual is compared with a range of amounts of extracellular DNMT RNA detected in said bodily fluid in populations of animals known to be free from a premalignant, neoplastic, or malignant disease. In particularly preferred aspects of this embodiment, comparison of DNMT RNA is further made to a reference RNA extracted, amplified, and detected from said bodily fluid, wherein said reference RNA is not DNMT RNA, but preferably an RNA normally present in the bodily fluid of both healthy individuals and those with cancer. In another aspect, said reference RNA is not DNMT RNA, but is an RNA present in the bodily fluid of individuals with cancer.

The methods of the invention further provide ways to identify individuals having a DNMT over expressing malignancy or premalignancy, thereby permitting rational, informed treatment options to be used for making therapeutic decisions. In particular, the methods of the invention are useful in identifying individuals having a premalignancy or malignancy that might benefit from a DNA methylation-directed therapy such as anti-methylation agents or antisense agents, either alone or administered with therapeutically-effective amounts of other chemotherapeutic or anticancer drugs. This aspect of the invention is particularly useful in identifying individuals for chemopreventive therapies, whether said therapy is directed at the methylation process or not.

Another advantageous use for the methods of the invention is to provide a marker for assessing the adequacy of anticancer therapy, including surgical intervention, chemotherapy, or radiation therapy, administered preventively or palliatively, or for determining whether additional or more advanced therapy is required. The invention therefore provides methods for developing a prognosis in such patients.

The methods of the invention also allows identification or analysis of DNMT RNA, either qualitatively or quantitatively, in the blood or other bodily fluid of an individual, most preferably a human who has completed therapy, as an early indicator of relapsed cancer, impending relapse, or treatment failure.

Specific preferred embodiments of the present invention will become evident from the following more detailed description of certain preferred embodiments and the claims.

### DETAILED DESCRIPTION OF THE INVENTION

The invention provides methods for detecting DNMT RNA, such as DNMT1 RNA and DNMT3 RNA including DNMT3a RNA and DNMT3b RNA, in bodily fluids of an animal, most preferably a human, thereby enabling detection of cancerous or precancerous cells that overexpress DNMT in the human or animal.

In preferred embodiments of the methods of the invention, extracellular RNA containing DNMT RNA is extracted from a bodily fluid. This extracted RNA is then amplified, either after conversion into cDNA or directly, using in vitro amplification methods in either a qualitative or quantitative manner using primers or probes specific for the DNMT RNA or cDNA of interest. The amplified product or signal is then detected in either a qualitative or quantitative manner.

In the practice of the methods of the invention, DNMT RNA may be extracted from any bodily fluid, including but not limited to whole blood, plasma, serum, urine, effusions, ascitic fluid, saliva, cerebrospinal fluid, cervical secretions, vaginal secretions, endometrial secretions, gastrointestinal secretions, bronchial secretions including sputum, breast fluid, or secretions or washings or lavages, using, for example, extraction methods described in co-owned and co-pending U.S. patent application Ser. No. 09/155,152, the entire disclosure of which is hereby been incorporated by reference. In a preferred embodiment, the bodily fluid is either blood plasma or serum. It is preferred, but not required, that blood be processed soon after drawing, and preferably within three hours, as to minimize any nucleic acid degradation in the sample. In a preferred embodiment, blood is first collected by venipuncture and kept on ice until use. Preferably, within 30 minutes to one hour of drawing the blood, serum is separated by centrifugation, for example at 1100×g for 10 minutes at 4° C. When using plasma, the blood is not permitted to coagulate prior to separation of the cellular and acellular components. Serum or plasma can be frozen, most preferably at −70° C. after separation from the cellular portion of blood until further assayed. When using frozen blood plasma or serum, the frozen serum or plasma is rapidly thawed, for example in a 37° C. water bath, and RNA is extracted therefrom without delay, most preferably using a commercially-available kit (for example, Perfect RNA Total RNA Isolation Kit, obtained from Five Prime—Three Prime, Inc., Boulder, Colo.). Other methods of RNA extraction are further provided in co-owned and co-pending U.S. patent application Ser. No. 09/155,152, incorporated herein by reference in its entirety.

Following the extraction of RNA from a bodily fluid, a fraction of which contains DNMT mRNA, the DNMT mRNA or cDNA derived therefrom is amplified in vitro. Applicable amplification assays are detailed in co-owned and co-pending U.S. patent application Ser. No. 09/155,152, as herein incorporated by reference, and include but are not limited to reverse transcriptase polymerase chain reaction (RT-PCR), ligase chain reaction, DNA signal amplification methods including branched chain signal amplification, amplifiable RNA reporters, Q-beta replication, transcription-based amplification, boomerang DNA amplification, strand displacement activation, cycling probe technology, isothermal nucleic acid sequence based amplification, and other self-sustained sequence replication assays.

In preferred embodiments of the methods of the invention, DNMT mRNA is converted into cDNA using reverse transcriptase prior to in vitro amplification using methods known in the art. For example, a sample, such as 10 microL extracted serum RNA is reverse-transcribed in a 30 microL volume containing 200 Units of Moloney murine leukemia virus (MMLV) reverse transcriptase (Promega, Madison, Wis.), a reaction buffer supplied by the manufacturer, 1 mM dNTPs, 0.5 micrograms random hexamers, and 25 Units of RNAsin (Promega, Madison, Wis.). Reverse transcription is typically performed under an overlaid mineral oil layer to inhibit evaporation and incubated at room temperature for 10 minutes followed by incubation at 37° C. for one hour.

Alternatively, other methods well known in the art can be used to reverse transcribe DNMT RNA to cDNA, as provided in these references incorporated herein by reference in their entirety, or by oligodT or primer-specific methods of reverse transcription.

Amplification primers are specific for amplifying DNMT-encoding nucleic acid. In a preferred embodiment, amplification of DNMT1, DNMT3a, or DNMT3b is performed by RT-PCR, preferably as set forth in Robertson et al. (Nucleic Acids Res. 27.2291-2298, 1999), incorporated herein by reference in its entirety. In these embodiments, the preferred oligonucleotide primer sequences are as follows:

For DNMT1:

(DNMT1 sense; SEQ ID No.1)

| | |
|---|---|
| Primer 1: | 5′-GATCGAATTCATGCCGGCGCGTACCGCCCCAG-3′ |

(DNMT1 antisense; SEQ ID No.2)

| | |
|---|---|
| Primer 2: | 5′-ATGGTGGTTTGCCTGGTGC-3′. |

For DNMT3a:

(DNMT3a sense; SEQ ID No.3)

| | |
|---|---|
| Primer 3: | 5′-GGGGACGTCCGCAGCGTCACAC-3′. |

(DNMT3a antisense; SEQ ID No.4)

| | |
|---|---|
| Primer 4: | 5′-CAGGGTTGGACTCGAGAAATCGC-3′. |

For DNMT3b:

(DNMT3b sense; SEQ ID No.5)

| | |
|---|---|
| Primer 5: | 5′-CCTGCTGAATTACTCACGCCCC-3′ |

(DNMT3b antisense; SEQ ID No.6)

| | |
|---|---|
| Primer 6: | 5′-GTCTGTGTAGTGCACAGGAAAGCC-3′. |

In one example of a preferred embodiment of the invention, DNMT RNA is harvested from approximately 1.75 mL aliquots of serum or plasma, and RNA extracted therefrom using the Perfect RNA Total RNA Isolation Kit (Five Prime—Three Prime) or similar commercial extraction kit. From this extracted RNA preparation, 10 microL are then reverse transcribed to cDNA as described above. RT-PCR for the DNMT cDNA is performed using 5 microL of DNMT cDNA in a final volume of 50 microL in a reaction mixture containing 1U of Amplitaq Gold (Perkin Elmer Corp., Foster City, Calif.), a reaction buffer provided by the Amplitaq supplier, 1.5 mM MgCl₂, 200 microM each dNTP, and 10 picomoles each of appropriate primer as identified above (Primer 1 and 2 for DNMT1; Primer 3 and 4 for DNMT3a; Primer 5 and 6 for DNMT3b). The mixture is then amplified in a single-stage reaction in a thermocycler under a temperature profile consisting of an initial 2 minute incubation at 94° C., followed by 45 cycles of denaturation at 94° C. for 30 seconds, annealing at 58° C. for DNMT1 or 65° C. for DNMT3a and DNMT3b, each for 60 seconds, and extension at 72° C. for 60 seconds, followed by a final extension at 72° C. for 5 minutes. Detection of the amplified product is then achieved, for example, by gel electrophoresis through a 4% Tris-borate-EDTA (TBE) agarose gel, using ethidium bromide staining for visualization and identification of the product fragment. Alternatively, the amplified products may thereafter be hybridized to end-labeled oligonucleotide probes and detected, such as using the method of Robertson et al. (Nucleic Acids Res. 27: 2291-2298, 1999). Further, it will be understood that alternative amplification primers and parameters can be utilized using methods known in the art.

The invention provides for alternative methods of amplification of DNMT RNA or cDNA known in the art, including but not limited to the methods of Chen et al. (Int. J. Cancer 83: 10-14, 1999), or Saito et al. (Hepatology 33: 561-568, 2001), incorporated herein by reference in their entirety, and further including signal amplification methods as known in the art. Amplification methods can further be performed in qualitative or quantitative fashion using primers specific for an internal control sequence of a reference RNA, such as glyceraldehyde-3-phosphate dehydrogenase or beta-actin, as described in said references, wherein said controls are RNA present in the bodily fluid of both healthy individuals and individuals with cancer.

In a particularly preferred embodiment, DNMT RNA or cDNA is amplified in a quantitative amplification reaction. Quantitative amplification of DNMT RNA or cDNA is particularly advantageous because this method enables statistically-based discrimination between patients with neoplastic disease and populations without neoplasm, including normal individuals. Using these methods, quantitative distributions of DNMT RNA in bodily fluids such as blood plasma or serum are established in populations with neoplastic diseases, and in normal populations. Using this population information, the amount of extracellular DNMT RNA in an individual is compared with the range of amounts of extracellular DNMT RNA in said populations, resulting in a determination of whether the detected amount of extracellular DNMT RNA in an individual indicates that the individual has a premalignant, neoplastic or malignant disease, or has a predisposition to developing such a disease.

In alternative preferred embodiments, amplified products can be detected using other methods, including but not limited to gel electrophoresis; capillary electrophoresis; ELISA or modifications thereof, such as amplification using biotinylated or otherwise modified primers; nucleic acid hybridization using specific, detectably-labeled probes, such as fluorescent-, radioisotope-, or chromogenically-labeled probe; Southern blot analysis; Northern blot analysis; electrochemiluminescence; reverse dot blot detection; and high-performance liquid chromatography. Furthermore, detection may be performed in either a qualitative or quantitative fashion.

PCR product fragments produced using the methods of the invention can be further cloned into recombinant DNA replication vectors using standard techniques. RNA can be produced from cloned PCR products, and in some instances the RNA expressed thereby, using the TnT Quick Coupled Transcription/Translation kit (Promega, Madison, Wis.) as directed by the manufacturer.

The methods of the invention as described above can be performed in like manner for detecting DNMT mRNA from other bodily fluids, including but not limited to whole blood, urine, effusions, ascitic fluid, saliva, cerebrospinal fluid, cervical secretions, vaginal secretions, endometrial secretions, gastrointestinal secretions, breast fluid or secretions, and bronchial secretions including sputum, and from washings or lavages. Whereas fractionation of the bodily fluid into its cellular and non-cellular components is not required for the practice of the invention, the non-cellular fraction may be separated, for example, by centrifugation or filtration of the bodily fluid.

The methods of the invention are thereby useful in the practice of a diagnostic method for detecting DNMT mRNA in an animal, most preferably a human at risk for developing or who has developed a premalignant, neoplastic or malignant disease consisting of cells over expressing DNMT mRNA. The invention further provides a method of identifying humans at risk for developing, or who have developed premalignancies or cancer, including but not limited to cancers of the breast, prostate, ovary, lung, cervix, colon, rectum, stomach, liver, pancreas, bladder, endometrium, kidney, brain, skin including squamous cell cancer and malignant melanoma, and esophagus, as well as premalignancies and carcinoma in-situ including but not limited to prostatic intraepithelial neoplasia (PIN), cervical dysplasia and cervical intraepithelial neoplasia (CIN), bronchial dysplasia, atypical hyperplasia of the breast, ductal carcinoma in-situ, colorectal adenoma, atypical endometrial hyperplasia, and Barrett's esophagus.

The diagnostic methods and advantageous applications of the invention can be performed using a diagnostic kit as provided by the invention, wherein the kit includes primers specific for DNMT cDNA synthesis or in vitro amplification or both, and/or specific probes for detecting DNMT RNA, cDNA or in vitro amplified DNA fragments or amplified signals thereof. The kit may further include methods and reagents for extracting DNMT RNA from an extracellular bodily fluid, wherein the bodily fluid includes but is not limited to plasma or serum.

The inventive methods have significant utility in assigning and monitoring non-specific therapies, including anti-neoplastic therapies such as chemotherapy, radiation, and surgery, or specific therapies such as antisense therapies and methylation-directed therapeutic agents. The inventive methods are also useful for monitoring response, relapse, and prognosis of DNMT producing neoplastic diseases. Of particular value, the invention allows a determination that a therapy is therapeutically indicated even in cases of premalignancy, early cancer, occult cancer or minimum residual disease are present. Thus, the invention permits selection of patients for said therapies or monitoring of therapeutic intervention, including chemoprevention, when tumor burden is low or when malignancy has not yet developed.

The invention further enables DNMT RNA to be evaluated in blood plasma or serum or other bodily fluid in combination with detection of other tumor-associated or tumor-derived RNA or DNA, including hypermethylated or aberrantly methylated DNA, in a concurrent or sequential fashion, such as in a multiplexed assay or in a chip-based assay, thereby increasing the sensitivity or efficacy of the assay in the detection or monitoring of neoplastic diseases, or in monitoring and evaluating aberrant DNA methylation processes.

The methods of the invention and preferred uses for the methods of the invention are more fully illustrated in the following Example. This Example illustrates certain aspects of the above-described method and advantageous results. This Example is shown by way of illustration and not by way of limitation.

### EXAMPLE 1

A 37 year old man with a family history of colorectal cancer undergoes a cancer predisposition screening test by providing a blood plasma sample for a multiplexed assay that includes evaluation of the plasma for DNMT RNA. DNMT RNA is evaluated by the methods of the invention in a quantitative manner as described. In addition, other tumor-associated nucleic acids, including K-ras DNA and hTERT RNA, are evaluated by the multiplexed assay. The assay indicates DNMT RNA is present in the plasma at levels higher than expected in the normal population. In addition, the multiplexed assay is positive for mutated K-ras oncogene present in the plasma, but negative for hTERT RNA. Overall, the assay results would indicate an increased predisposition for neoplasia. The man would subsequently undergo a conventional colonoscopy, and have two adenoma detected and removed. As the patient is considered at high risk for developing colorectal neoplasia in the future, the man would start a chemopreventive drug therapy regimen. Serial evaluation of quantitative DNMT RNA levels in plasma is undertaken to evaluate response to the chemoprevention regimen. DNMT RNA levels demonstrate progressive decline into the range for a normal population during the treatment period, indicating a good response to therapy.

This example demonstrates use of the invention for detection and monitoring of neoplasia, and determining predisposition to neoplasia. Futhermore, the example demonstrates use of the invention in monitoring response to a chemoprevention regimen.

What is claimed is:

1. A method of detecting DNA methyltransferase (DNMT) RNA in blood plasma or serum from a human or animal for detecting, diagnosing, monitoring, treating, or evaluating a neoplastic disease that expresses DNMT RNA, the method comprising the steps of:
a) extracting mammalian RNA from blood plasma or serum;
b) amplifying or signal amplifying a portion of the extracted RNA or cDNA prepared therefrom, wherein said fraction comprises DNMT RNA, and wherein amplification is performed in either a qualitative or quantitative fashion using primers or probes specific for DNMT RNA or cDNA; and
c) detecting the amplified DNMT RNA or cDNA product or signal.

2. A method of detecting DNA methyltransferase (DNMT) RNA in a bodily fluid, from a human or animal for detecting, diagnosing, monitoring, treating, or evaluating a neoplastic disease that expresses DNMT RNA, the method comprising the steps of:
a) extracting mammalian RNA from a bodily fluid;
b) amplifying or signal amplifying a portion of the extracted RNA or cDNA prepared therefrom, wherein said fraction comprises DNMT RNA, and wherein amplification is performed in either a qualitative or quantitative fashion using primers or probes specific for DNMT RNA or cDNA; and
c) detecting the amplified DNMT RNA or cDNA product or signal.

3. The method of claim 2, wherein the bodily fluid is whole blood, blood plasma, serum, urine, effusions, ascites, saliva, cerebrospinal fluid, cervical secretions, endometrial secretions, gastrointestinal secretions, bronchial secretions, or breast fluid, lavages, or aspirations.

4. The method of claim 1, wherein the amplification in step (b) is performed by an RNA amplification method that amplifies the RNA directly or wherein the RNA is first reverse transcribed to cDNA, whereby the cDNA is amplified, wherein the amplification method is reverse transcriptase polymerase chain reaction, ligase chain reaction, signal amplification, amplifiable RNA reporters, Q-beta replication, transcription-based amplification, isothermal nucleic acid sequence based amplification, self-sustained sequence replication assays, boomerang DNA amplification, strand displacement activation, or cycling probe technology.

5. The method of claim 2, wherein the amplification in step (b) is performed by an RNA amplification method that amplifies the RNA directly or wherein the RNA is first reverse transcribed to cDNA, whereby the cDNA is amplified, wherein the amplification method is reverse transcriptase polymerase chain reaction, ligase chain reaction, signal amplification, amplifiable RNA reporters, Q-beta replication, transcription-based amplification, isothermal nucleic acid sequence based amplification, self-sustained sequence replication assays, boomerang DNA amplification, strand displacement activation, or cycling probe technology.

6. The method of claim 1, wherein detection of amplified product in step (c) is performed using a detection method that is gel electrophoresis, capillary electrophoresis, ELISA detection using biotinylated or otherwise modified primers, labeled fluorescent or chromogenic probes, Southern blot analysis, Northern blot analysis, electrochemilummescence, reverse dot blot detection, or high-performance liquid chromatography.

7. The method of claim 2, wherein detection of amplified product in step (c) is performed using a detection method that is gel electrophoresis, capillary electrophoresis, ELISA detection using biotinylated or otherwise modified primers, labeled fluorescent or chromogenic probes, Southern blot analysis, Northern blot analysis, electrochemiluminescence, reverse dot blot detection, or high-performance liquid chromatography.

8. A method of identifying a human or animal having DNA methyltransferase (DNMT) RNA expressing cells or tissue, the method comprising the steps of:
a) extracting mammalian RNA from a bodily fluid;
b) amplifying or signal amplifying a portion of the extracted RNA or cDNA prepared therefrom, wherein said fraction comprises DNMT RNA, and wherein amplification is performed in a qualitative or quantitative fashion using primers or probes specific for DNMT RNA or cDNA; and
c) detecting the amplified DNMT RNA or cDNA product or signal.

9. The method of claim 8, wherein the DNMT expressing cells or tissue comprise a malignant or premalignant cell or tissue.

10. The method of claim 8, wherein a predisposition of developing a malignancy or premalignancy is thereby determined.

11. The method of claim 8, wherein the human or animal has a malignancy or premalignancy.

12. A method according to claim 1, wherein a predisposition for malignancy is determined.

13. A method according to claim 2, wherein a predisposition for malignancy is determined.

14. A method according to claim 1, further comprising the step of selecting a human or animal for a therapy when the amplified DNMT RNA or cDNA product or signal is detected.

15. A method according to claim 2, further comprising the step of selecting a human or animal for a therapy when the amplified DNMT RNA or cDNA product or signal is detected.

16. A method according to claim 8, wherein the cells or tissue are characterized by hypermethylated DNA.

17. A method for detecting DNA methyltransferase (DNMT) RNA, or cDNA reverse-transcribed therefrom, comprising the steps of extracting RNA comprising DNMT RNA from blood plasma or serum, with or without converting said RNA to cDNA, hybridizing said RNA or cDNA to a detectably-labeled probe specific for DNMT RNA or cDNA, and detecting hybridization of DNMT RNA or cDNA with the detectably-labeled probe.

18. A method for detecting DNA methyltransferase (DNMT) RNA, or cDNA reverse-transcribed therefrom, comprising the steps of extracting RNA comprising DNMT RNA from a bodily fluid, with or without converting said RNA to cDNA, hybridizing said RNA or cDNA to a detectably-labeled probe specific for DNMT RNA or cDNA, and detecting hybridization of DNMT RNA or cDNA with the detectably-labeled probe.

19. A method according to claim 1, wherein the method comprises the additional step of quantitatively or qualitatively comparing the product of DNMT RNA in the plasma or serum of a human subject to the product of DNMT RNA in the plasma or serum from a plurality of humans with or without known malignancy or premalignancy.

20. A method according to claim 2, wherein the method comprises the additional step of quantitatively or qualitatively comparing the product of DNMT RNA in the bodily fluid of a human to the product of DNMT RNA in the bodily fluid from a plurality of humans with or without known malignancy or premalignancy.

21. A method for detecting a plurality of mammalian RNA species in a bodily fluid from a human or animal, wherein one mammalian RNA species is a DNA methyltransferase (DNMT) RNA species, the method comprising the steps of:
a) extracting mammalian RNA from the bodily fluid of a human or animal, wherein said extracted RNA comprises a plurality of mammalian RNA species, wherein one RNA species is DNMT RNA;
b) amplifying or signal amplifying concurrently or sequentially at least one of said plurality of RNA or cDNA produced therefrom, wherein one of said RNA is DNMT RNA, to thereby produce an amplified product, wherein amplification is performed qualitatively or quantitatively using primers or probes specific for each RNA species; and
c) detecting the amplified product produced from every amplified RNA species or cDNA produced therefrom.

22. The method of claim 21, wherein the bodily fluid is blood plasma or serum.

23. The method of claim 21, wherein detection of a plurality of RNA species in the bodily fluid is indicative or predictive of malignancy or premalignancy, wherein one RNA species is DNMT RNA.

24. A diagnostic kit, comprising DNA methyltransferase (DNMT) RNA or cDNA specific amplification primers or probes.

25. The method of claim 1, wherein DNMT RNA is DNA methyltransferase I RNA, DNA methyltransferase 3a RNA, or DNA methyltransferase 3b RNA.

26. The method of claim 2, wherein DNMT RNA is DNA methyltransferase 1 RNA, DNA methyltransferase 3a RNA, or DNA methyltransferase 3b RNA.

27. The method of claim 8, wherein DNMT RNA is DNA methyltransferase 1 RNA, DNA methyltransferase 3a RNA, or DNA methyltransferase 3b RNA.

28. The method of claim 17, wherein DNMT RNA is DNA methyltransferase 1 RNA, DNA methyltransferase 3a RNA, or DNA methyltransferase 3b RNA.

29. The method of claim 18, wherein DNMT RNA is DNA methyltransferase 1 RNA, DNA methyltransferase 3a RNA, or DNA methyltransferase 3b RNA.

30. The method of claim 21, wherein DNMT RNA is DNA methyltransferase 1 RNA, DNA methyltransferase 3a RNA, or DNA methyltransferase 3b RNA.

31. The method of claim 24, wherein DNMT RNA is DNA methyltransferase 1 RNA, DNA methyltransferase 3a RNA, or DNA methyltransferase 3b RNA.
